(19) United States
(12) Patent Application Publication
(10) Pub. No.: US 2025/0114568 A1
(43) Pub. Date: Apr. 10, 2025

(54) EXTERNAL SUTURELESS FIXATION DEVICE FOR ELEMENTS WHICH EXTEND INTO THE BODY

(71) Applicants: University of Pittsburgh - Of the Commonwealth System of Higher Education, Pittsburgh, PA (US); UPMC, Pittsburgh, PA (US)

(72) Inventors: Trent D. Emerick, Mars, PA (US); Sergio Hickey, Pocatello, ID (US)

(21) Appl. No.: 18/729,408

(22) PCT Filed: Jan. 16, 2023
(86) PCT No.: PCT/US2023/010865

§ 371 Date: Jul. 16, 2024

Related U.S. Application Data
(60) Provisional application No. 63/300,160, filed on Jan. 17, 2022.

(51) Int. Cl. A61M 25/02

(52) U.S. Cl. CPC A61M 25/02

(57) ABSTRACT

A device for externally securing one or more extending elements which extend from external to a patient's body to internal to the patient's body includes a first member including one or more channels, wherein each of the one or more channels is dimensioned to position a length of one of the one or more extending elements at least partially therein, and one or more engagement members, and a second member including one or more cooperating engagement members configured to form a cooperating engagement with the one or more engagement members of the first member so that a surface of the second member is positioned adjacent to the one or more channels of the first member when the second member is engaged with the first member

### CROSS-REFERENCE TO RELATED APPLICATIONS

This application claims benefit of U.S. Provisional Patent Application Ser. No. 63/300,160, filed Jan. 17, 2022, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

In various medical procedures it is desirable to implant extending members or elements such as wires or conduits (for example, catheters) which extend percutaneously from inside the body to outside of the body after implantation. For example, implantable nerve stimulators are commonly used to treat a variety of chronic pain conditions. These nerve stimulators can, for example, be placed in the epidural space of the spinal cord for diffuse pain or along a peripheral nerve for a specific nerve injury. These devices are often permanently implanted inside the body through a surgical procedure. Before committing to a permanent surgery for the implanted nerve stimulator, however, a nonsurgical trial is often undertaken to determine if the patient is an appropriate candidate for the permanent surgical procedure. The trial is commonly approximately one week in duration but may be 10-21 days for spinal cord temporary stimulators, and up to 90 days for peripheral nerve stimulation systems. The temporary trial stimulator wire is placed through a hollow needle along the nerve, and then the needle is removed, leaving only the wire along the nerve, with the other end of the wire protruding through the skin and attached to an external battery pack.

Traditional safety measures to prevent movement of trial simulator wires include use of tape, sutures, fixation device, and/or knots in the wires. However, such traditional safety measure too often fail. Sutures, for example, commonly fail, by breaking, tearing the skin, and/or causing worsening pain. Failure in such traditional safety measures can result in migration of one or more wire inside the body. Such migration can occur to an extent that the wire(s) is/are no longer visible outside the body. It has been experienced that a wire or wires may, for example, migrate from the lumbar region into the high thoracic spine. Such an event requires an urgent surgical intervention to remove the wires from the epidural space.

It is thus desirable to develop additional or alternative safety measures to stabilize wires or conduits which extend into the body (for example, percutaneously).

### SUMMARY

In one aspect, a device for externally securing one or more extending elements which extend from external to a patient's body to internal to the patient's body includes a first member including one or more channels. Each of the one or more channels is dimensioned to position a length of one of the one or more extending elements at least partially therein. The first member further includes one or more engagement members. The device further includes a second member including one or more cooperating engagement members configured to form a cooperating engagement with the one or more engagement members of the first member so that a surface of the second member is positioned adjacent to the one or more channels of the first member when the second member is engaged with the first member. The one or more extending elements may, for example, pass into the patient's body percutaneously, orally, etc. In a number of embodiments, the one or more extending elements pass into the patient's body percutaneously.

In a number of embodiments, the first member includes a sufficient number of channels so that at least two different lengths of one or more of the one or more of extending elements can be positioned within the one or more channels after looping of the one or more extending elements. Each of the one or more channels may, for example, have a width that is approximately equivalent to or less than a diameter of one of the one or more extending elements to be positioned therein.

The one or more cooperating engagement members of the second member may, for example, be configured to slidably engage with the one or more engagement members of the first member. The one or more cooperating engagement members of the second member may, for example, be configured to engage with the one or more engagement members of the first member via a snap fit, threading, a bayonet-type connection, a luer-lock-type connection, etc. In a number of embodiments, the second member is connected to the first member via a hinging connection.

In a number of embodiments, the device further includes an antibacterial composition. In a number of embodiments, the device further includes an adhesive layer on a surface thereof to assist in attaching the device to the patient's body.

In another aspect, a method of securing one or more extending elements which extend from external to a patient's body to internal to the patient's body includes placing a device in operative connection with the one or more extending elements. The device includes a first member including one or more channels, wherein each of the one or more channels is dimensioned to position a length of one of the one or more extending elements at least partially therein, and one or more engagement members. The device further includes a second member including one or more cooperating engagement members configured to form a cooperating engagement with the one or more engagement members of the first member so that a surface of the second member is positioned adjacent the one or more channels of the first member when the second member is engaged with the first member. The method further includes attaching the device to the patient's body. The one or more extending elements may, for example, pass into the body percutaneously.

As described above, the first member may include a sufficient number of channels so that at least two different lengths of one or more of the one or more of extending elements can be positioned within the one or more channels after looping of the one or more extending elements. Each of the one or more channels may, for example, have a width that is approximately equivalent to or less than a diameter of one of the one or more extending elements to be positioned therein.

In a number of embodiments, the one or more cooperating engagement members of the second member are configured to slidably engage with the one or more engagement members of the first member. In a number of embodiments, the one or more cooperating engagement members of the second member are configured to engage with the one or more engagement members of the first member via a snap fit, threading, a bayonet-type connection, a luer-lock-type connection, etc. The second member may, for example, be connected to the first member via a hinging connection.

In a number of embodiments, the device further includes an antibacterial composition. In a number of embodiments, the device is attached to the skin of the patient via an adhesive.

The method may further include placing a dressing over the device. The dressing may, for example, include a transparent portion positioned over the device when the dressing in placed in operative connection with and over the device.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a lateral superior isometric view of a representative example of a device hereof with a second member of the device disconnected from a first or base member thereof.

FIG. 2 illustrates a frontal orthographic view of the device of FIG. 1 in a disconnected state with wires (shown in cross-section) seated in channels of the first member.

FIG. 3 illustrates a lateral orthographic view of the device of FIG. 1 in a disconnected state.

FIG. 4 illustrates a lateral superior isometric view of the device of FIG. 1 in a disconnected state.

FIG. 5 illustrates a superior orthographic view of the device of FIG. 1 in a disconnected state with wires positioned in channels of the first member.

FIG. 6 illustrates an inferior orthographic view of the device of FIG. 1 in a disconnected state.

FIG. 7 illustrates a superior lateral isometric view of the device of FIG. 1 in a disconnected state.

FIG. 8 illustrates a superior lateral isometric view of the device of FIG. 1 with the second member of the device partially operatively connected or coupled to the first member or base.

FIG. 9A illustrates a superior lateral isometric view of the device of FIG. 1 with the second member of the device operatively connected or coupled to the first member or base with wires positioned in channels of the first member.

FIG. 9B illustrates a frontal orthographic view of the device of FIG. 1 with the second member of the device operatively connected or coupled to the first member or base with wires (shown in cross-section) positioned in channels of the first member.

FIG. 9C illustrates a frontal orthographic view of the another embodiment of a device hereof similar to the device of FIG. 1 with a single wire (shown in cross-section) positioned in channels of the first member of the device.

FIG. 10A illustrates a frontal orthographic view of another embodiment of a device hereof in which the second member is connected to the first member or base via a living hinge with the second member positioned in in a disconnected, uncoupled, or open state.

FIG. 10B illustrates a frontal orthographic view of the device of FIG. 10A in a connected, coupled, or closed state.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a channel” includes a plurality of such channels and equivalents thereof known to those skilled in the art, and so forth, and reference to “the channel” is a reference to one or more such channels and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

In a number of embodiments hereof, a fixation device or system attaches to one or more extending wires or similar extending elements which extend from external to the body to internal to the body (for example, percutaneously extending element(s)). The device, systems, and methods here may, for example, operate to limit or prevent movement of the wire(s) or other extending element(s) and/or other safety concerns. The device, systems, and methods hereof stabilize the wire(s) or other extending element(s) without use of sutures. Devices, systems, and/or methods hereof may, for example, be used in connection with wires of a wireless nerve stimulator system (for example, such as those available from Stimwave Technologies or other companies with similar wireless technology, SPR SPRINT® PNS (peripheral nerve stimulator) or other wireless systems such as the Medtronic INTELLIS™ system or other comparable spinal cord stimulator trial systems.). The devices, systems, and methods hereof may, however, be used broadly in connection with any extending elements such as wires, conduits, tubes, catheters etc. For example, the devices, systems, and methods hereof may also be used in connection with temporary wireless external pacemaker wires, deep brain stimulation wires, temporary percutaneous, surgical drainage tubes, etc.

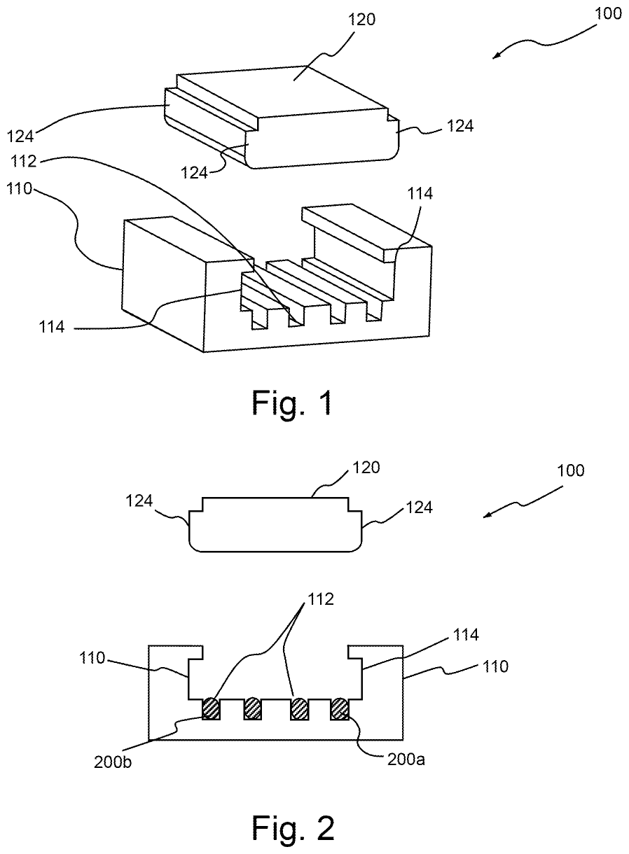

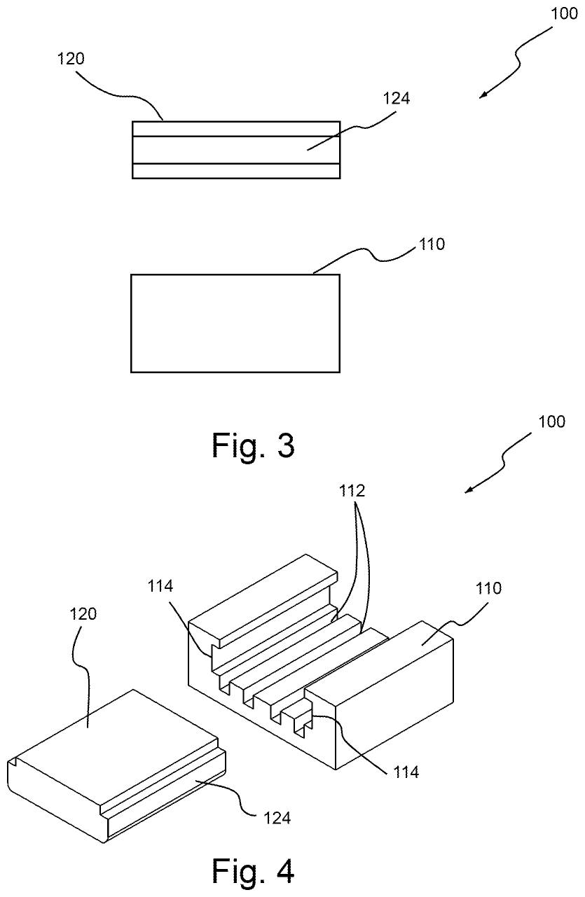

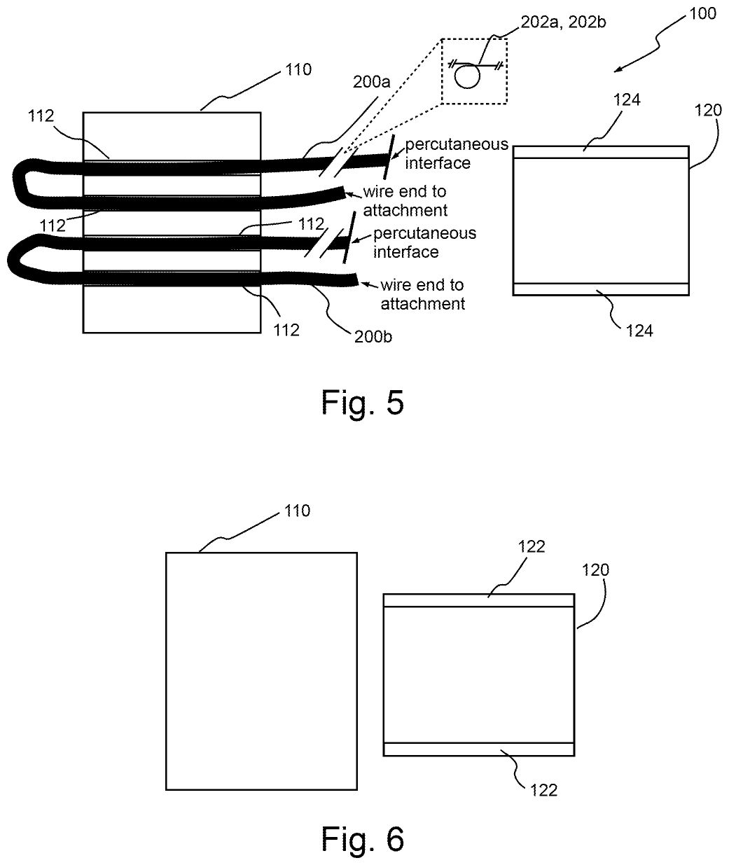

FIGS. 1 through 9B illustrate an embodiment of a device or system 100 hereof for use in connection with, for example, extending elements such as representative, temporary nerve stimulator wires 200a and 200b (see FIGS. 2, 5, 9A and 9B). In the illustrated embodiment of FIGS. 1 through 9B, device 100 includes a first or base member 110 and a second or cooperating member 120. First member 110 includes extending channels, grooves, or seatings 112 in which wires 200a and 200b may be seated as illustrated in FIGS. 2 and 5.

First member 110 and second member 120 may, for example, be formed from polymeric materials (for example, via a molding, printing, and/or other polymer processing methodology). In a number of studied embodiments, devices 100 were 3-D printed. In a number of embodiments, at least a portion of device 100 (for example, at least a portion of the surfaces of first member 110 or channels 112 thereof or second member 120) may include antibacterial properties or include an antibacterial material/composition thereon. Such an antibacterial material or composition may be used to prevent the spread of skin flora bacteria from tracking along wires 200a and 200b and into the body (for example, into the epidural space or the final location of such wires). Common skin bacteria that can be neutralized with antibacterial compositions include, but are not limited to Staphylococcus aureus (MSSA, MRSA) and Staphylococcus epidermidis. Antibacterial coatings or compositions use in connection with catheters may, for example, be used in connection with device 100 hereof. Suitable antibacterial compositions include, but are not limited to, chlorhexidine/silver sulfadiazine, silver, 5-fluorouracil, vancomycin, benzalkonium chloride, teicoplanin, miconazole/rifampicin, minocycline, minocycline/rifampin, quaternary ammonium compositions and phosphonium compositions. Antibacterial compositions may, for example, be coated on or incorporated into polymeric materials used in forming devices 100 hereof either physically (for example, via mixing as additive) or chemically.

Similar to practices used in connection with dressings for various catheters such as for central line catheters (for example, a peripherally inserted central catheter or PICC), various practices may be used in connection with device 100 to facilitate comfort and/or sterility during use thereof. For example, a gauze pad (for example, a 4×4 inch pad) may be placed between the skin and device 100 for the duration of the trial to prevent tissue irritation or abrasions between the skin and device 100. A dressing or protective cover (for example, a transparent film dressing such as the 3M™ TEGADERM™ dressing available from 3M of Saint Paul, Minnesota) may be applied over the device as well as over a portion of wires 200a and 200b (including, any strain relief section thereof as described herein). Such a dressing can, for example, provide a waterproof, sterile barrier to contaminants while providing visibility and increasing patient comfort.

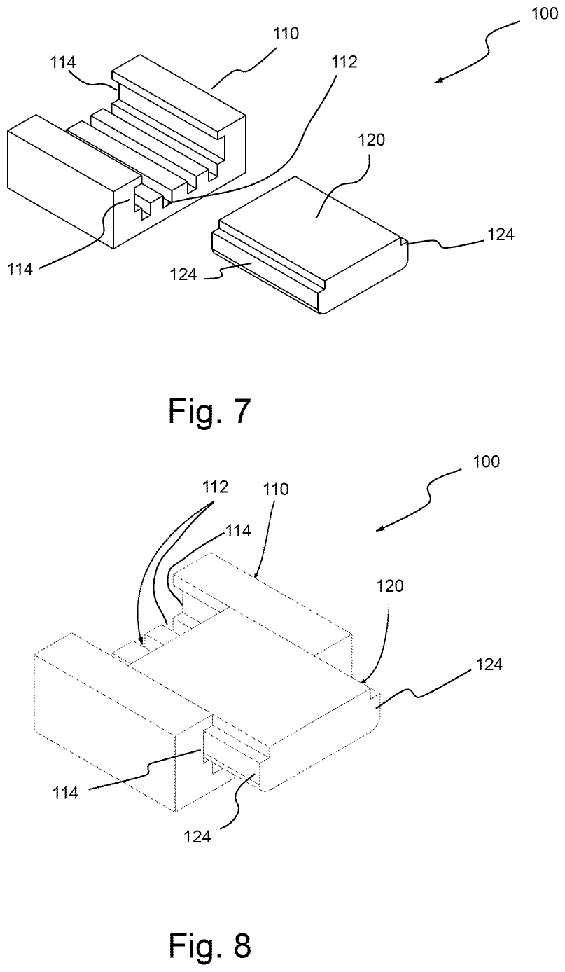

In the illustrated embodiment of FIGS. 1A through 9B, second member 120 is slidably engageable with first member 110. Second member 120 may, for example, include one or more cooperating engagement members which cooperate with one or more engagement members of first member 110 to connect or couple the two members. For example, second member 120, in the illustrated embodiment, includes one or more laterally extending projections or flanges 124 which cooperate with one or more extending channels 114 of first member 110 when second member 120 is aligned with and slid into engagement with first member 110 (see, for example, FIGS. 7 through 9B).

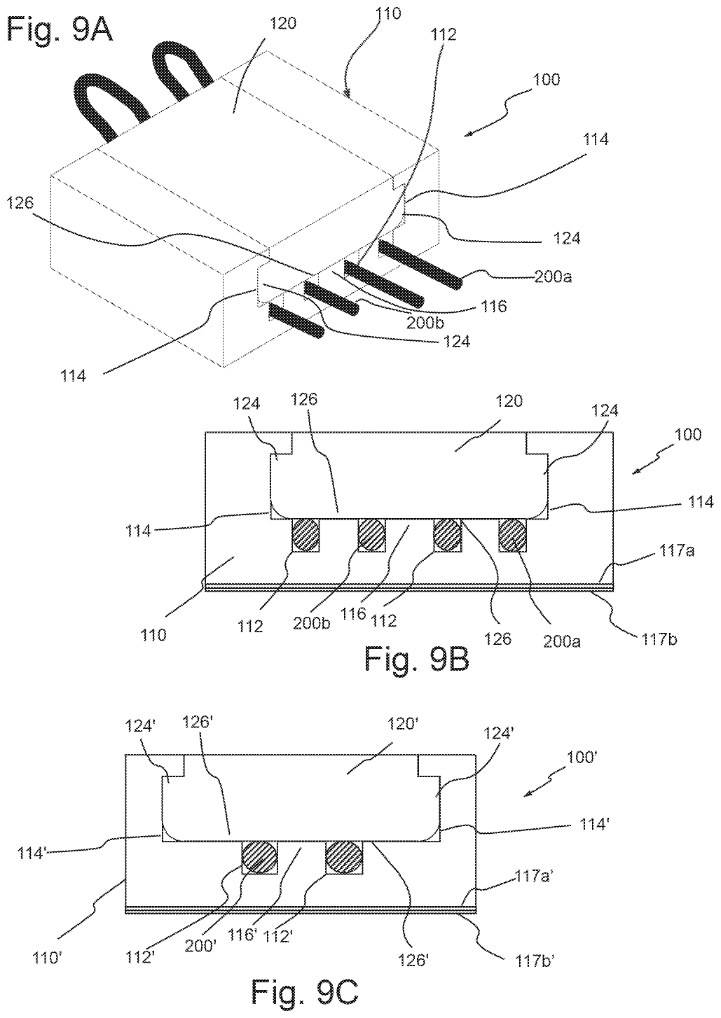

As illustrated in FIG. 5, temporary stimulator wires 200a and 200b (or other extending elements) may be seated within channel, groove, or seating 112 of first member 110 before second member 120 is engaged with first member 110. In the illustrated embodiment (see, FIGS. 5 and 9A), each of temporary stimulator wires 200a and 200b is extended from its respective percutaneous interface (or other interface via which wires 200a and 200b enter the body) to be seated in and extend through one of channels 112. Each of wires 200a and 200b may then be looped back to extend in the opposite direction so that a second length thereof passes through another one of channels 112, before second member 120 is slidably engaged with first member 110. A relief section 202a and 202b (for example, one or more coils or loops of wire) between device 100 and a percutaneous interface can be provided for tension or strain relief as illustrated in FIG. 5. Tension or strain relief sections 202a and 202b may, for example, be taped to the skin, (between the percutaneous skin exit site/interface and device 100) and can, for example, be helpful if the patient significantly moves in a certain direction so that the distal end of the wire inside the patient body does not pull out as the loops or coils of sections 202a and 202b accommodate any pulling or tugging. Looping back or returning of wires 200a and 200b through device 100 as illustrated in FIGS. 5 and 9A can assist in limiting or preventing movement/migration of wires 200a and 200b.

Channels 112 may, for example, be dimensioned to maintain contact with wires 200a and 200b. In a number of embodiments, channels 112 have a width that is approximately equal to (for example, within 5% or within 1%) or slightly less than (for example, 5% or 1% less than) a diameter/width of wires 200a and 200b to ensure a relatively tight seating of wires 200a and 200b therein. Wires 200a and 200b (or other elements) may thus be held in place via an abutting friction fit. Wires 200a and 200b such a titanium or alloy wires typically used in neurostimulation (or other elements) are typically somewhat compressible to deform when seated within channels 112. Additionally, a lower surface 126 (see, for example, FIG. 9A and 9B) of second member 120 may be adjacent to the upper surface of channels 112 (for example, spaced less than a diameter of the smallest of one of wires 200a and 200b or other extending element(s) from surface 116 of first member 110 in which channels 112 are formed), abutting the upper surface of channels 112, or abutting wires 200a and 200b to limit or prevent movement thereof relative to device 100 once second member 120 connected to first member 110. Upon seating of wires 200a and 200b in channels 112 and engagement of second member 120 with first member 110, device 100 can be attached to the patient via, for example, adhesive tape, bandaging, via an adhesive layer 117a (see FIG. 9B) on a lower or bottom surface of first member 110 or via adhesive dressing film as described above. As known in the art, an adhesive surface such as adhesive layer 117a can be protected by a removable cover layer or film 117b until time for attachment to a patient.

Device 100 may, in a number of embodiments, be suitably sized or dimensioned for use with temporary nerve stimulator wires 200a and 200b. Once again, device 100 can be sized or dimensioned for use with many types of extending elements such as percutaneously extending elements. Moreover, more or less than four channels 112 may be provided as required for the number of extending element to be seated and any desired looping through channels as described above. For example, FIG. 9C illustrates an embodiment of a device 100′ including two channels 112′. In other respects, device 100′ is formed in generally the same manner as device 100, and components of device 100′ are numbered similarly to like components of device 100 with the addition of the designation “‘’” to the reference numeral. Further, the widths of the channels may vary within the device or between devices to seat differently sized extending elements.

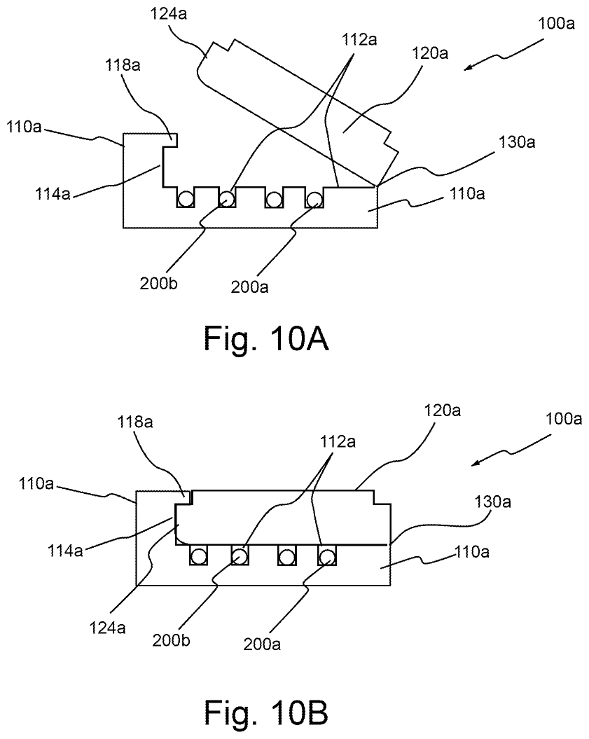

The manner in which a second or cooperating member hereof engages first or base member hereof can vary as known in the closure mechanism arts. For example, FIGS. 10A and 10B illustrate another embodiment of a device 100a hereof. In the embodiment of device 100a, second or cooperating member 120a is connected to first or base member 110a via an integral or living hinge 130a (that is, a thin, flexible hinge) connecting first member 110a to second member 120a. As illustrated in FIG. 10A, second member 120a may be rotated or pivoted relative to first member 110a via hinge 130a. Rotating or pivoting second member 120a to a closed position as illustrated in FIG. 10B results in a “snap-fit” closure wherein a cooperating engagement member in the form of a lateral extending flange 124a causes deformation of a flange 118a and seating of flange 124a within channel 114a. As discussed above in connection with device 100, wires 200a and 200b (or other extending elements) can be positioned or seated in channels 112a of first member 110a. Other types of connections such as threaded connections, bayonet-type connections, luer-lock-type connections, etc. between the first member and the second member of device hereof may be used in various embodiments hereof.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A device for externally securing one or more extending elements which extend from external to a patient's body to internal to the patient's body, comprising:
a first member comprising one or more channels, wherein each of the one or more channels is dimensioned to position a length of one of the one or more extending elements at least partially therein, and one or more engagement members, and
a second member comprising one or more cooperating engagement members configured to form a cooperating engagement with the one or more engagement members of the first member so that a surface of the second member is positioned adjacent to the one or more channels of the first member when the second member is engaged with the first member.

2. The device of claim 1 wherein the one or more extending elements pass into the patient's body percutaneously.

3. The device of claim 1 wherein the first member includes a sufficient number of channels so that at least two different lengths of one or more of the one or more of extending elements can be positioned within the one or more channels after looping of the one or more extending elements.

4. The device of claim 1 wherein the one or more cooperating engagement members of the second member are configured to slidably engage with the one or more engagement members of the first member.

5. The device of claim 1 wherein the one or more cooperating engagement members of the second member are configured to engage with the one or more engagement members of the first member via a snap fit.

6. The device of claim 1 wherein the second member is connected to the first member via a hinging connection.

7. The device of claim 1 further comprising an antibacterial composition.

8. The device of claim 1 wherein each of the one or more channels has a width that is approximately equivalent to or less than a diameter of one of the one or more extending elements to be positioned therein.

9. The device of claim 1 further comprising an adhesive layer on a surface thereof to assist in attaching the device to the patient's body.

10. A method of securing one or more extending elements which extend from external to a patient's body to internal to the patient's body, comprising:
placing a device in operative connection with the one or more extending elements, the device comprising a first member comprising one or more channels, wherein each of the one or more channels is dimensioned to position a length of one of the one or more extending elements at least partially therein, and one or more engagement members, and a second member comprising one or more cooperating engagement members configured to form a cooperating engagement with the one or more engagement members of the first member so that a surface of the second member is positioned adjacent to the one or more channels of the first member when the second member is engaged with the first member, and
attaching the device to the patient's body.

11. The method of claim 10 wherein the one or more extending elements pass into the body percutaneously.

12. The method of claim 10 wherein the first member includes a sufficient number of channels so that at least two different lengths of one or more of the one or more of extending elements can be positioned within the one or more channels after looping of the one or more extending elements.

13. The method of claim 10 wherein the one or more cooperating engagement members of the second member are configured to slidably engage with the one or more engagement members of the first member.

14. The method of claim 10 wherein the one or more cooperating engagement members of the second member are configured to engage with the one or more engagement members of the first member via a snap fit.

15. The method of claim 10 wherein the second member is connected to the first member via a hinging connection.

16. The method of claim 10 wherein the device further comprises an antibacterial composition.

17. The method of claim 10 wherein each of the one or more channels has a width that is approximately equivalent to or less than a diameter of one of the one or more extending elements to be positioned therein.

18. The method of claim 10 wherein the device is attached to the skin of the patient via an adhesive.

19. The method of claim 10 further comprising placing a dressing over the device.

20. The method of claim 19 wherein the dressing comprises a transparent portion positioned over the device.

21. A system, comprising:
one or more extending elements which extend from external to a patient's body to internal to the patient's body; and
a device for externally securing the one or more extending elements comprising a first member comprising one or more channels, wherein each of the one or more channels is dimensioned to position a length of one of the one or more extending elements at least partially therein, and one or more engagement members, and
a second member comprising one or more cooperating engagement members configured to form a cooperating engagement with the one or more engagement members of the first member so that a surface of the second member is positioned adjacent to the one or more channels of the first member when the second member is engaged with the first member.
